(19) United States
(12) Patent Application Publication
(10) Pub. No.: US 2013/0190869 A1
(43) Pub. Date: Jul. 25, 2013

(54) Method of Forming a Prosthesis From a User Kit

(71) Applicant: Wendy A. Smith, West St. Paul (CA)

(72) Inventor: Wendy A. Smith, West St. Paul (CA)

(21) Appl. No.: 13/357,271

(22) Filed: Jan. 24, 2012

(51) Int. Cl. A61F 2/52; B29C 39/20

(52) U.S. Cl. 623/7; 264/129

(57) ABSTRACT

A prosthesis, for example a breast or a finger prosthesis, is formed for mating with a first body portion of a user such that the prosthesis is symmetrical with a second body portion of the user. The prosthesis is formed at a manufacturing location using castings of the first and second body portions which the user creates themselves at a user location separate from the manufacturing location using a user casting kit. The user casting kit includes casting material, skin colour samples and any additional tools required for the subsequent manufacture of a custom prosthesis without the user being required to leave the user location.

### FIELD OF THE INVENTION

The present invention relates to a casting kit enabling a user to perform the casting step in the subsequent manufacturing process of a custom prosthesis.

### BACKGROUND

Reconstructive surgery is known to be performed subsequent to a mastectomy in some instance to improve body symmetry to the patient; however, this option is not available to many patients for various reasons. In this instance, an external prosthesis is commonly used to provide body symmetry to the patient. External prostheses which are available typically include either: i) mass produced prostheses; or ii) custom prostheses. Mass produced prosthesis are generally not well fitted to the user and thus are uncomfortable to wear for many users. Custom prostheses are also disadvantageous in many instances as conventional techniques for producing custom prostheses are costly, have limited availability, and require considerable interaction between the user and the manufacturer in order to be customized to the user.

### SUMMARY OF THE INVENTION

According to one aspect of the invention there is provided a method of forming a prosthesis for mating with a first body portion of a user such that the prosthesis is symmetrical with a second body portion of the user, the method comprising:

delivering a user kit to the user at a user location in which the user kit comprises a casting material and colour samples;

instructing the user to apply the casting material to both the first body portion and the second body portion to form respective casting portions;

instructing the user to make a colour sample selection from the colour samples of the kit;

receiving the casting portions and colour sample selection delivered from the user at the user location to a manufacturing location separate from the user location;

molding a replica of the first body portion and a replica of the second body portion of the user using the casting portions at the manufacturing location;

shaping a body of the prosthesis at the manufacturing location such that the body of the prosthesis is symmetrical with the replica of the second body portion when mated with the replica of the first body portion; and

colouring the prosthesis according to the colour sample selection made by the user.

The user casting kit effectively enables multiple users at different respective user locations to each be provided a prosthesis which is customized to them, but which is manufactured at a common manufacturing location by using the castings formed by the users not otherwise possible without the user casting kit. Accordingly a custom prosthesis can be made available to a larger number of people at a more reasonable cost than otherwise possible.

In the illustrated embodiment the prosthesis comprises a breast prosthesis such that the first body portion comprises a chest area of the user and the second body portion comprises a breast of the user. Alternatively, the prosthesis may comprise a finger prosthesis or a prosthesis for any other body part which could be fashioned from a corresponding symmetrical body part.

In the instance of a breast prosthesis, preferably the colour samples in the kit relate to skin colour and areola colour such that the method includes instructing the user to make a first colour sample selection relating to skin colour and a second colour sample selection relating to areola colour, and colouring the prosthesis according to the first and second colour sample selections made by the user.

When there are also provided areola size samples in the kit preferably the method includes instructing the user to make an areola size sample selection from the areola size samples, and colouring the prosthesis according to the areola size sample selection made by the user.

The user is preferably instructed to fit a bra over the casting material as the casting material sets for subsequently receiving said bra with the casting portions and colour sample selection delivered from the user and shaping a body of the prosthesis such that the body of the prosthesis fits within said bra.

When an adhesive allergy test strip is provided in the kit and the user is instructed to apply the adhesive allergy test strip to skin of the user to determine if the user has an allergic reaction, the method may additionally include optionally applying an adhesive to the body of the prosthesis if the user does not have an allergic reaction to the adhesive allergy test strip.

When the kit includes tattoo samples, the user may make a tattoo selection from the tattoo samples of the kit such that the method further includes colouring the prosthesis to include a tattoo according to the tattoo sample selection made by the user.

According to a second aspect of the present invention there is provided a method of forming a prosthesis for mating with a first body portion of a user such that the prosthesis is symmetrical with a second body portion of the user, the method comprising:

receiving a user kit at a user location in which the user kit comprises a casting material and colour samples;

applying the casting material to both the first body portion and the second body portion to form respective casting portions;

selecting a skin colour selection from the colour samples corresponding to skin tones of the user; and

delivering the casting and skin color selection from the user location to a manufacturing location separate from the user location such that a replica of the first body portion and a replica of the second body portion can be molded at the manufacturing location and such that a body of the prosthesis can be shaped at the manufacturing location to mate with the replica of the first body portion and be symmetrical with the replica of the second body portion with colouring applied according to the colour sample selection made by the user.

When the method includes fitting a bra over the casting material as the casting material cures, preferably the bra is delivered with the casting portions and colour sample selection from the user location to the manufacturing location for use in shaping a body of the prosthesis such that the body of the prosthesis fits within said bra. Preferably a waterproof sheet is positioned between the bra and casting material during the casting process.

When the method includes the user making a first colour sample selection relating to skin colour and a second colour sample selection relating to areola colour, preferably the first and second colour samples are delivered from the user location to the manufacturing location for use in colouring the prosthesis according to the first and second colour sample selections.

Similarly, when the method includes the user making an areola size sample selection from the areola size samples and/or making a tattoo selection from the tattoo samples of the kit, the areola size sample selection is preferably delivered from the user location to the manufacturing location for use in colouring the prosthesis according to the areola size sample selection made by the user and according to the tattoo sample selection made by the user.

In the preferred embodiment the colour samples each comprise a silicon member including a colour layer and a transparent layer overtop of the colour layer in which the method includes delivering the silicon member of the corresponding skin colour selection with the casting from the user location to the manufacturing location.

Preferably the kit also includes a release agent which is applied to the first and second body portions of the user prior to applying the casting material to the first and second body portions.

In some embodiments, the kit may include a camera. In this instance the method includes the user taking a photo of the second body portion and delivering the photo with the casting portions from the user location to the manufacturing location.

In the preferred embodiment, the prosthesis comprises a breast prosthesis such that the first body portion comprises a chest area of the user and the second body portion comprises a breast of the user, wherein the kit further comprises a release agent, return packaging, casting material, plastic sheeting, a measuring tape, areola size samples, skin colour samples, and areola colour samples in which the colour samples each comprise a silicon member including a colour layer and a transparent layer overtop of the colour layer. In this instance the method further includes the steps of:

i) selecting a first colour sample relating to skin colour of the user;

ii) selecting a second colour sample relating to areola colour of the user;

iii) selecting an areola size sample relating to areola size of the user;

iv) measuring about a torso of the user using the measuring tape;

v) applying the release agent to the first and second body portions of the user prior to applying the casting material to the first and second body portions;

vi) fitting a bra over the casting material as the casting material cures; vii) positioning a waterproof sheet between the bra and casting material; and

viii) delivering said bra, the silicon members of the selected first and second colour samples, the areola size selection, and the measurement of the torso with the casting portions from the user location to the manufacturing location for use in shaping a body of the prosthesis such that the body of the prosthesis fits within said bra and for use in colouring the prosthesis according to the first and second colour samples and the areola size sample.

One embodiment of the invention will now be described in conjunction with the accompanying drawings in which:

### BRIEF DESCRIPTION OF THE DRAWINGS

FIG. 1 is a schematic representation of the components of the user casting kit; and

FIGS. 2A through 2E illustrate various steps of the casting process in which:

FIG. 2A illustrates the application of the release agent;

FIG. 2B illustrates the application of a first strip of casting material;

FIG. 2C illustrates the application of all of the casting material;

FIG. 2D illustrates the application of a waterproof sheet and bra overtop of the casting material; and

FIG. 2E illustrates removal of the completed casting from the user.

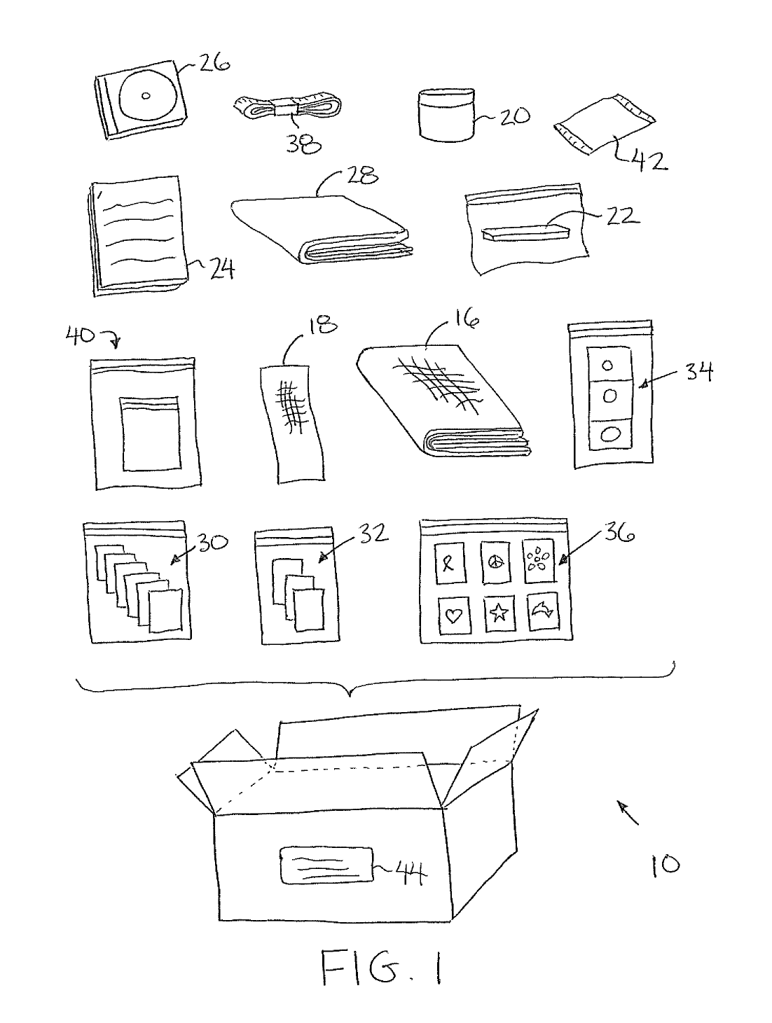

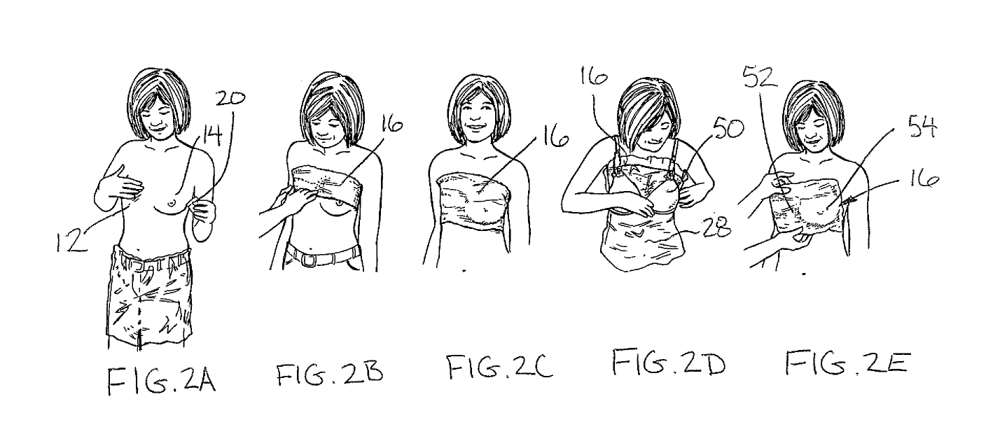

In the drawings like characters of reference indicate corresponding parts in the different figures.

### DETAILED DESCRIPTION

Referring to the accompanying figures, there is illustrated a user casting kit generally indicated by reference numeral 10. The kit 10 is a tool for assisting a manufacturer to make a custom prosthesis for a user where the user produces their own castings for forming the prosthesis.

The kit is particularly suited for use with a prosthesis of the type which mates with a first body portion 12 of the user and is arranged to be symmetrical with a second body portion 16 of the user. In the illustrated embodiment, the prosthesis comprises a breast prosthesis in which the first body portion comprises a chest area to one side of the user while the second body portion comprises the breast of the user on the other side of the chest area.

In an alternative embodiment in which the prosthesis comprises a finger, the first body portion in this instance comprises a knuckle area on the hand where the prosthesis is to be worn while the second body portion comprises the corresponding finger on the other hand of the user.

The kit is packaged as a single item delivered from the manufacturer at a manufacturing location to a user at a respective user location separate from the manufacturing area. Accordingly, the kit is well suited for being delivered to the residence of the user such that the user can conduct the casting within the privacy of their own residence, while the subsequent manufacturing of the prosthesis using the castings is accomplished at a separate manufacturing location.

The kit 10 includes various components for performing the casting. A primary component of the kit are plaster bandages including main plaster bandages 16 for performing the casting as well as smaller test plaster bandages 18 for testing the casting process prior to forming the actual castings. In each instance, the plaster bandages comprise a textile material with an open weave which is coated with plaster which becomes activated when mixed with water. The kit is typically available in three different sizes in which each different kit size comprises different sizes of plaster bandages 16 for performing the casting.

The kit further includes a suitable release agent 20, for example petroleum jelly, which is applied to the skin prior to application of the casting material to protect the skin of the user and aid in releasing of the casting from the user once the casting is set.

The kit also includes a sticky-back sample adhesive in the form of an adhesive allergy test strip 22 to determine if the user is allergic to adhesive which is optionally available on the prosthesis. To perform the test, the user initially unwraps the test sample strip 22 and applies it to the skin of the user which has been cleaned and is free of any moisturizer or creams. Once the test strip has been applied to the skin in proximity to the prosthesis area, the test strip is covered with plastic wrap and worn for 24 hours to determine if there is any reaction. Upon subsequent removal of the test strip, a determination is made as to whether the user is allergic to the adhesive depending upon whether or not the skin becomes itchy or irritated. Where there is a reaction, no adhesive can be applied to the subsequently manufactured prosthesis. Where there is no reaction, the user has the option of requesting adhesive being applied to the prosthesis or not. The adhesive decision is returned from the user to the manufacturer with the castings.

Paper instructions 24 and a DVD 26 with instructions thereon are additionally provided in the kit to instruct the user through the various stages of the casting process and through various selections to be made prior to or subsequent to the casting to be returned with the casting to the manufacturer to assist in manufacturing the prosthesis.

The instructions include initially testing the casting process using the test plaster bandages 18. In this instance, the user applies an even layer of petroleum jelly to a small area in proximity to the prosthesis area. The entire skin area to be in contact with the plaster strips must be covered. To begin casting, a test strip is dipped into warm water and the excess water is gently squeezed out prior to application of the strip to the chest area. The casting material can be stretched onto the user and is smoothed out using fingertips to conform to the body contours of the user prior to wetting and applying a subsequent strip. Subsequent strips are overlapped with previous applied strips in the order of 1 inch or greater along the full length thereof. Once the user is comfortable with the process, the test strips can be removed and the casting process begun. In the instance of a breast prosthesis, the release agent is initially applied to the entire chest area including both the first and second body portions. Prior to wetting the plaster, protective plastic sheets 28 provided in the kit are used to protect the floor and the clothing of the user by tucking the top edge of one of the sheets into the waist of the pants of the user. One of the protective sheets 28 is used as described below subsequent to the positioning of the casting material on the user.

Once a sufficient application of release agent is applied to the skin of the user, including both above and below the breast area, to both sides past the mid-line of the torso, and below the underarm area, the user can then begin applying the main plaster bandages 16. Once again, the plaster strips are wetted and then applied by stretching onto the user with each strip being smoothed to contour the body of the user prior to application of the next bandage strip with a similar overlap of approximately one inch along the full length.

The bandages are applied to fully cover the chest area including the first and second body portions across the full width of the torso from the mid-line of the torso at one side to the mid-line of the torso at the opposing side of the user. Immediately after applying the casting material and prior to curing of the casting material, one of the protective sheets 28 in the form of a waterproof plastic sheeting is applied overtop of the casting material and smoothed into place such that the user can put on their bra 50 in the usual manner. Once the bra has been properly fitted into place, the user should remain upright and standing while the plaster sets. The curing of the plaster forming the casting material initially releases heat felt by the user. Once the plaster material hardens and the heat has gone away, the cast can be carefully removed from the user and set aside. Typically, the casting material should dry for a period in the range of 48 hours to be sufficiently hardened before being returned to the manufacturing location from the user location.

In addition to returning the casting material which defines a first casting portion 52 of the first body portion and a second casting portion 54 of the second body portion respectively, the user must make several additional selections to be returned together with the casting portions. To perform these selections, the kit also includes a plurality of skin colour samples 30 and areola colour samples 32. Each of the colour samples comprises a flat silicone member formed of the same material as the prosthesis to be manufactured to ensure a true representation of the colour. More particularly, each colour sample comprises a colour layer of silicone and a transparent layer of silicone overtop of the coloured layer in accordance with the manufacturing process of the prosthesis.

In the illustrated embodiment six skin colour samples are provided corresponding to a range of different skin tones and three colour samples relating to a range of areola colours are provided. The user selects one skin colour sample and one areola colour sample which best correspond with the second body portion of the user. These selected samples are included in the package with the casting portions returned to the manufacturer. Furthermore, the kit includes three areola size samples 34 corresponding to different areola sizes for selection by the user. The areola size samples can also be returned with the casting portions if desired.

The kit further includes six tattoo samples 36 again in the form of silicone members representing the actual material of the prosthesis to be formed in which a different graphical image in the form of a tattoo is provided on each sample. The user optionally can select one of the tattoo samples to be returned together with the casting portions for being represented on the subsequent prosthesis to be manufactured.

A tape measure 38 is also enclosed within the kit to permit the user to take a circumference measurement about the torso along with any other additional measurements which may be useful for the manufacturer. These measurements are also returned with the casting to the manufacturing location.

The kit further includes sealable plastic bags 40 for use by the user. The bra which was used for fitting over the casting material is placed in one of the bags according to the instructions provided to the user for being returned to the manufacturer together with the casting portions. The other bag receives the various size and colour selections by the user. In addition to the bags 40, the casting kit further includes a desiccant material 42 included in the package delivered to the user for the user's subsequent use to be enclosed in the package with the casting material for return to the manufacturer. The user is also provided with suitable packaging including a return shipping label 44 and instructions for the user to pack the casting portions together with the various selections for return to the manufacturer.

At the manufacturing location, the manufacturer receives the casting portions together with the colour sample selections, the size selections and any other selections including tattoo selections or the adhesive selection for example. The manufacturer molds a replica of the first body portion and a replica of the second body portion using the casting portions made by the user. The replicas can be used at the manufacturing location for subsequently shaping a body of the prosthesis such that the body mates with the first body portion and is symmetrical with the second body portion when it is mated with the first body portion. The bra provided by the user can be used by the manufacturer to ensure that the prosthesis fits with the bra that was used for forming the castings.

In addition to forming the shape of the prosthesis according to the castings and the bra provided by the user, the manufacturer also colours the prosthesis according to the skin colour sample, the areola colour sample, the areola size sample, and the tattoo sample selected by the user and returned to the manufacturer. Adhesive can be applied to a portion of the prosthesis which mates with the first body portion of the user, only if the user has indicated a negative reaction to the allergy test strip and has further indicated the desire for adhesive to be provided.

In further embodiments, the kit may also be provided with a camera, for example a disposable type camera, such that the user can take a photo of the first and second body portions. The photo and the camera can be returned from the user to the manufacturing location for additional use by the manufacturer for customizing the prosthesis to the user.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method of forming a prosthesis for mating with a first body portion of a user such that the prosthesis is symmetrical with a second body portion of the user, the method comprising:
delivering a user kit to the user at a user location in which the user kit comprises a casting material and colour samples;
instructing the user to apply the casting material to both the first body portion and the second body portion to form respective casting portions;
instructing the user to make a colour sample selection from the colour samples of the kit;
receiving the casting portions and colour sample selection delivered from the user at the user location to a manufacturing location separate from the user location;
molding a replica of the first body portion and a replica of the second body portion of the user using the casting portions at the manufacturing location;
shaping a body of the prosthesis at the manufacturing location such that the body of the prosthesis is symmetrical with the replica of the second body portion when mated with the replica of the first body portion; and
colouring the prosthesis according to the colour sample selection made by the user.

2. The method according to claim 1 wherein the prosthesis comprises a breast prosthesis such that the first body portion comprises a chest area of the user and the second body portion comprises a breast of the user.

3. The method according to claim 2 including providing colour samples in the kit relating to skin colour and areola colour, instructing the user to make a first colour sample selection relating to skin colour and a second colour sample selection relating to areola colour, and colouring the prosthesis according to the first and second colour sample selections made by the user.

4. The method according to claim 3 including providing areola size samples in the kit, instructing the user to make an areola size sample selection from the areola size samples, and colouring the prosthesis according to the areola size sample selection made by the user.

5. The method according to claim 2 including instructing the user to fit a bra over the casting material as the casting material sets, receiving said bra with the casting portions and colour sample selection delivered from the user, and shaping a body of the prosthesis such that the body of the prosthesis fits within said bra.

6. The method according to claim 1 including providing an adhesive allergy test strip in the kit, instructing the user to apply the adhesive allergy test strip to skin of the user to determine if the user has an allergic reaction, and applying an adhesive to the body of the prosthesis if the user does not have an allergic reaction to the adhesive allergy test strip.

7. The method according to claim 1 including providing tattoo samples in the kit, instructing the user to make a tattoo selection from the tattoo samples of the kit, and colouring the prosthesis to include a tattoo according to the tattoo sample selection made by the user.

8. A method of forming a prosthesis for mating with a first body portion of a user such that the prosthesis is symmetrical with a second body portion of the user, the method comprising:
receiving a user kit at a user location in which the user kit comprises a casting material and colour samples;
applying the casting material to both the first body portion and the second body portion to form respective casting portions;
selecting a skin colour selection from the colour samples corresponding to skin tones of the user; and
delivering the casting and skin color selection from the user location to a manufacturing location separate from the user location such that a replica of the first body portion and a replica of the second body portion can be molded at the manufacturing location and such that a body of the prosthesis can be shaped at the manufacturing location to mate with the replica of the first body portion and be symmetrical with the replica of the second body portion with colouring applied according to the colour sample selection made by the user.

9. The method according to claim 8 wherein the prosthesis comprises a breast prosthesis such that the first body portion comprises a chest area of the user and the second body portion comprises a breast of the user.

10. The method according to claim 9 including fitting a bra over the casting material as the casting material cures.

11. The method according to claim 10 including delivering said bra with the casting portions and colour sample selection from the user location to the manufacturing location for use in shaping a body of the prosthesis such that the body of the prosthesis fits within said bra.

12. The method according to claim 9 including positioning a waterproof sheet between the bra and casting material.

13. The method according to claim 9 wherein the colour samples in the kit relate to skin colour and areola colour and wherein the method includes the user making a first colour sample selection relating to skin colour and a second colour sample selection relating to areola colour and delivering the first and second colour samples from the user location to the manufacturing location for use in colouring the prosthesis according to the first and second colour sample selections.

14. The method according to claim 9 wherein the kit includes areola size samples and wherein the method includes the user making an areola size sample selection from the areola size samples and delivering the areola size sample selection from the user location to the manufacturing location for use in colouring the prosthesis according to the areola size sample selection made by the user.

15. The method according to claim 8 including providing tattoo samples in the kit, instructing the user to make a tattoo selection from the tattoo samples of the kit, and colouring the prosthesis to include a tattoo according to the tattoo sample selection made by the user.

16. The method according to claim 8 wherein the colour samples each comprise a silicon member including a colour layer and a transparent layer overtop of the colour layer and wherein the method includes delivering the silicon member of the corresponding skin colour selection with the casting from the user location to the manufacturing location.

17. The method according to claim 8 wherein the kit includes a release agent and wherein the method includes applying the release agent to the first and second body portions of the user prior to applying the casting material to the first and second body portions.

18. The method according to claim 8 wherein the kit includes an adhesive allergy test strip and wherein the method includes applying the adhesive allergy test strip to skin of the user to determine if the user has an allergic reaction, making an adhesive selection if the user does not have an allergic reaction to the adhesive allergy test strip and delivering the adhesive selection with the casting portions from the user location to the manufacturing location for use in applying an adhesive to the body of the prosthesis according to the adhesive selection.

19. The method according to claim 8 wherein the kit includes a camera and wherein the method includes the user taking a photo of the second body portion and delivering the photo with the casting portions from the user location to the manufacturing location.

20. The method according to claim 8 wherein the prosthesis comprises a breast prosthesis such that the first body portion comprises a chest area of the user and the second body portion comprises a breast of the user and wherein the kit further comprises a release agent, return packaging, casting material, plastic sheeting, a measuring tape, areola size samples, skin colour samples, and areola colour samples in which the colour samples each comprise a silicon member including a colour layer and a transparent layer overtop of the colour layer, the method further comprising:
selecting a first colour sample relating to skin colour of the user;
selecting a second colour sample relating to areola colour of the user;
selecting an areola size sample relating to areola size of the user;
measuring about a torso of the user using the measuring tape;
applying the release agent to the first and second body portions of the user prior to applying the casting material to the first and second body portions;
fitting a bra over the casting material as the casting material cures;
positioning a waterproof sheet between the bra and casting material; and
delivering said bra, the silicon members of the selected first and second colour samples, the areola size selection, and the measurement of the torso with the casting portions from the user location to the manufacturing location for use in shaping a body of the prosthesis such that the body of the prosthesis fits within said bra and for use in colouring the prosthesis according to the first and second colour samples and the areola size sample.
